(19) United States
(12) Patent Application Publication
(10) Pub. No.: US 2016/0256324 A1
(43) Pub. Date: Sep. 8, 2016

(54) LASER TREATMENT APPARATUS

(71) Applicant: Kabushiki Kaisha Topcon, Itabashi-ku (JP)

(72) Inventor: Hideharu SUZUKI, Nerima-ku (JP)

(73) Assignee: Kabushiki Kaisha Topcon, Itabashi-ku (JP)

(21) Appl. No.: 14/967,537

(22) Filed: Dec. 14, 2015

(30) Foreign Application Priority Data
Mar. 5, 2015 (JP) 2015-043201

(51) Int. Cl. A61F 9/008

(52) U.S. Cl. CPC A61F 9/00821; A61F 9/00825; A61F 2009/00891; A61F 2009/00863; A61F 2009/00868; A61F 2009/00897

(57) ABSTRACT

According to one embodiment, a laser treatment apparatus is configured to apply laser treatment to a patient's eye, and includes an irradiation system, an illumination system, an imaging system, an image acquisition unit, and a composite image presenting unit. The irradiation system irradiates the patient's eye with laser beams emitted from a light source. The illumination system illuminates the patient's eye with slit light. The imaging system guides the slit light returning from the patient's eye to an imaging device. The image acquisition unit acquires a first image that represents a region of the patient's eye including the illumination field of the slit light. The composite image presenting unit presents a composite image of the first image acquired by the image acquisition unit and a second image of the patient's eye based on output of the imaging device.

### CROSS-REFERENCE TO RELATED APPLICATION

This application is based upon and claims the benefit of priority from Japanese Patent Application No. 2015-043201, filed May 3, 2015; the entire contents of which are incorporated herein by reference.

### FIELD

Embodiments described herein relate generally to a laser treatment apparatus used in the ophthalmologic field.

### BACKGROUND

A laser treatment apparatus is used to treat a variety of eye diseases and conditions. For example, to treat a certain kind of glaucoma, the corner (angulus iridocornealis: a part including the trabeculae and being located between the cornea and the iris) is irradiated with a laser beam to form the outflow pathway of aqueous humor. Besides, panretinal photocoagulation is performed in the treatment of retinal diseases such as diabetic retinopathy.

There has been a known laser treatment apparatus configure to aim a predetermined pattern of aiming beams (array of a plurality of spots) at a treatment area, and then irradiate the area with a predetermined pattern of laser beams (see, for example, Japanese Translation of PCT International Application Publication No. 2009-514564, Japanese Unexamined Patent Application Publication No. 2014-54462, Japanese Patent No. 5166454).

Generally, a doctor performs an ophthalmologic laser treatment while observing a patient's eye with a slit lamp microscope. In the observation using a slit lamp microscope, the slit is not fully opened so that the patient does not feel dazzling. This means that the operator performs aiming and laser irradiation while observing a small area of the patient's eye. Accordingly, it is hard for the operator who is not trained in laser treatment to identify the site of the patient's eye being aimed at and irradiated with laser beams only through the observation under the slit illumination. Even the expert finds it difficult to do this depending on the conditions of the patient's eye and observation site.

### SUMMARY

Embodiments are intended to provide a technology for easily identifying a site of the patient's eye, which is being aimed at or irradiated with laser beams.

According to one embodiment, a laser treatment apparatus is configured to apply laser treatment to a patient's eye, and includes an irradiation system, an illumination system, an imaging system, an image acquisition unit, and a composite image presenting unit. The irradiation system irradiates the patient's eye with laser beams emitted from a light source. The illumination system illuminates the patient's eye with slit light. The imaging system guides the slit light returning from the patient's eye to an imaging device. The image acquisition unit acquires a first image that represents a region of the patient's eye including the illumination field of the slit light. The composite image presenting unit presents a composite image of the first image acquired by the image acquisition unit and a second image of the patient's eye based on output of the imaging device.

According to the embodiment, it is possible to easily identify a site of the patient's eye, which is being aimed at or irradiated with laser beams.

### BRIEF DESCRIPTION OF THE DRAWINGS

FIG. 1 is a schematic diagram illustrating an example of the configuration of a laser treatment apparatus according to an embodiment.

FIG. 2 is a schematic diagram illustrating an example of the configuration of the laser treatment apparatus of the embodiment.

FIG. 3 is a schematic diagram illustrating an example of the configuration of the laser treatment apparatus of the embodiment.

FIG. 4 is a schematic diagram illustrating an example of the operation of the laser treatment apparatus of the embodiment.

FIG. 5 is a schematic diagram illustrating an example of the configuration of a laser treatment apparatus according to another embodiment.

FIG. 6 is a schematic diagram illustrating an example of the configuration of a laser treatment apparatus according to a modification of the embodiment.

FIG. 7 is a schematic diagram illustrating an example of the configuration of a laser treatment apparatus according to still another embodiment.

FIG. 8 is a schematic diagram illustrating an example of the operation of the laser treatment apparatus of the embodiment.

FIG. 9 is a schematic diagram illustrating an example of the configuration of a laser treatment apparatus according to still another embodiment.

FIG. 10 is a schematic diagram illustrating an example of the configuration of the laser treatment apparatus of the embodiment.

### DETAILED DESCRIPTION

Referring now to the drawings, a laser treatment apparatus is described in some exemplary embodiments. The technologies disclosed in the above cited documents can be incorporated into the embodiments described below. The embodiments and modifications described below may be combined arbitrarily.

The following are definitions of directions. Here, a direction from the optical system of the apparatus toward a patient is defined as forward direction, and a direction opposite thereto is defined as backward direction. A horizontal direction perpendicular to the forward direction is defined as lateral direction (right-left direction). In addition, a direction perpendicular to both the forward-backward direction and the lateral direction is defined as vertical direction.

### First Embodiment

### Configuration

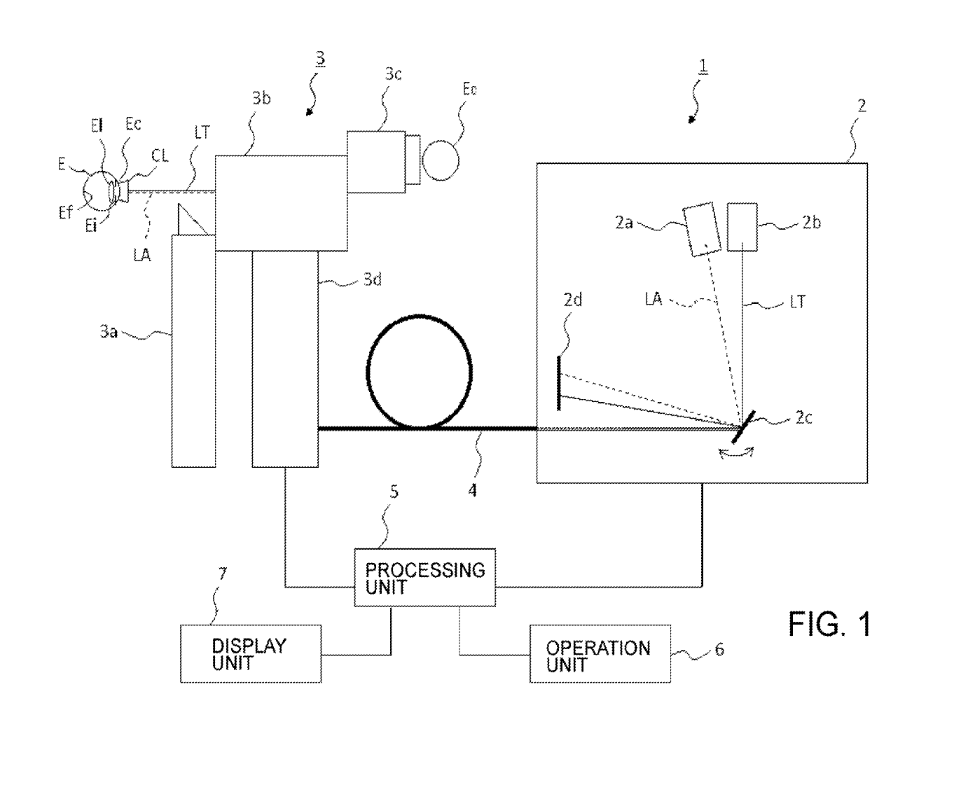

FIG. 1 illustrates an example of the configuration of a laser treatment apparatus 1 according to the first embodiment. The laser treatment apparatus 1 is used to treat a patient's eye E with laser therapy. The laser therapy is applied to the fundus Ef, the corner, and the like. The corner is a part where the cornea Ec is in contact with the iris Ei. In FIG. 1, the reference letter El denotes the crystalline lens.

The laser treatment apparatus 1 includes a light source unit 2, a slit lamp microscope 3, an optical fiber 4, a processing unit 5, an operation unit 6, and a display unit 7. The slit lamp microscope 3 may be replaced by a surgical microscope, an indirect ophthalmoscope, an intraocular observation device, or the like.

The light source unit 2 and the slit lamp microscope 3 are optically connected to each other via the optical fiber 4. The optical fiber 4 includes one or more light guides. The light source unit 2 and the processing unit 5 are connected to each other to transmit signals therebetween. The slit lamp microscope 3 and the processing unit 5 are connected to each other to transmit signals therebetween. The operation unit 6 and the processing unit 5 are connected to each other to transmit signals therebetween. The signals may be transmitted via a wired or wireless connection.

The processing unit 5 includes a computer that operates by the cooperation of hardware and software. The processing performed by the processing unit 5 is described later. The operation unit 6 includes various hardware keys and/or software keys such as a graphical user interface (GUI). Examples of the hardware key include: a button, a handle, a knob and the like provided on the slit lamp microscope 3; a keyboard, a pointing device (a mouse, a trackball, etc.) and the like provided in a computer (the processing unit 5, etc.) connected to the slit lamp microscope 3; and a foot switch, an operating panel and the like that are provided separately. The software key is, for example, displayed on a display device provided to the slit lamp microscope 3 or the computer.

### (Light Source Unit 2)

The light source unit 2 generates light to be irradiated to the fundus Ef. The light source unit 2 includes an aiming light source 2a, a treatment light source 2b, a galvanometer mirror 2c, and a light-shielding plate 2d. Incidentally, the light source unit 2 may include a member other than those illustrated in FIG. 1. For example, an optical element (lens, etc.) may be arranged immediately in front of the optical fiber 4 to make light generated by the light source unit 2 incident on the end face of the optical fiber 4.

### (Aiming Light Source 2a)

The aiming light source 2a generates an aiming beam LA for aiming at a site subjected to a laser treatment. Arbitrary light source may be used as the aiming light source 2a. For example, if the aiming is performed while a patient's eye E is being visually observed, a light source (a laser light source, a light emitting diode, etc.) that emits visible light recognizable by the operator's eye E₀ is used as the aiming light source 2a. On the other hand, if the aiming is performed while an image captured of the patient's eye E is being observed, a light source (a laser light source, a light emitting diode, etc.) that emits light of wavelengths at which an imaging device for capturing images has a sensitivity is used as the aiming light source 2a. The aiming beam LA is guided to the galvanometer mirror 2c. The processing unit 5 controls the operation of the aiming light source 2a.

### (Treatment Light Source 2b)

The treatment light source 2b emits a laser beam for treatment (treatment light LT). The treatment light LT may be either visible or invisible laser beam depending on the intended use. The treatment light source 2b may be a single laser light source or a plurality of laser light sources that emit laser beams of different wavelengths from each other. The treatment light LT is guided to the galvanometer mirror 2c. The processing unit 5 controls the operation of the treatment light source 2b.

### (Galvanometer Mirror 2c)

The galvanometer mirror 2c includes a mirror having a reflecting surface and an actuator configured to change the orientation of the mirror (the orientation of the reflecting surface). The aiming beam LA and the treatment light LT are designed to reach the same position on the reflecting surface of the galvanometer mirror 2c. Incidentally, the treatment light LT and the aiming beam LA may be collectively referred to as “irradiation light”. The orientation of the galvanometer mirror 2c (the reflecting surface) is changed to at least an orientation (for irradiation) to reflect the irradiation light toward the optical fiber 4, and an orientation (for stop) to reflect the irradiation light toward the light-shielding plate 2d. The processing unit 5 controls the operation of the galvanometer mirror 2c.

### (Light-Shielding Plate 2d)

If the galvanometer mirror 2c is arranged in the orientation for stop, the irradiation light reaches the light-shielding plate 2d. For example, the light-shielding plate 2d is in a form and/or made of a material that absorbs the irradiation light, and has a light-shielding property.

In the present embodiment, the aiming light source 2a and the treatment light source 2b each continuously generate light. Further, the galvanometer mirror 2c is arranged in the orientation for irradiation to irradiate the patient's eye E with the irradiation light. In addition, the galvanometer mirror 2c is arranged in the orientation for stop to stop irradiating the patient's eye E with the irradiation light.

In other embodiments, the aiming light source 2a and/or the treatment light source 2b may be configured to be capable of intermittently generating light. That is, the aiming light source 2a and/or the treatment light source 2b may be configured to be capable of generating pulse light. The pulse control therefor is performed by the processing unit 5. Such a configuration does not require the galvanometer mirror 2c and the light-shielding plate 2d.

### (Slit Lamp Microscope 3)

The slit lamp microscope 3 is an apparatus used to observe the anterior segment and the fundus Ef of the patient's eye E. More specifically, the slit lamp microscope 3 is an ophthalmologic apparatus that illuminates the patient's eye E with slit light, and provides a magnified image of the illumination field for observation. Incidentally, the “observation” includes either or both of observation through an ocular lens and observation of an image captured by the imaging device.

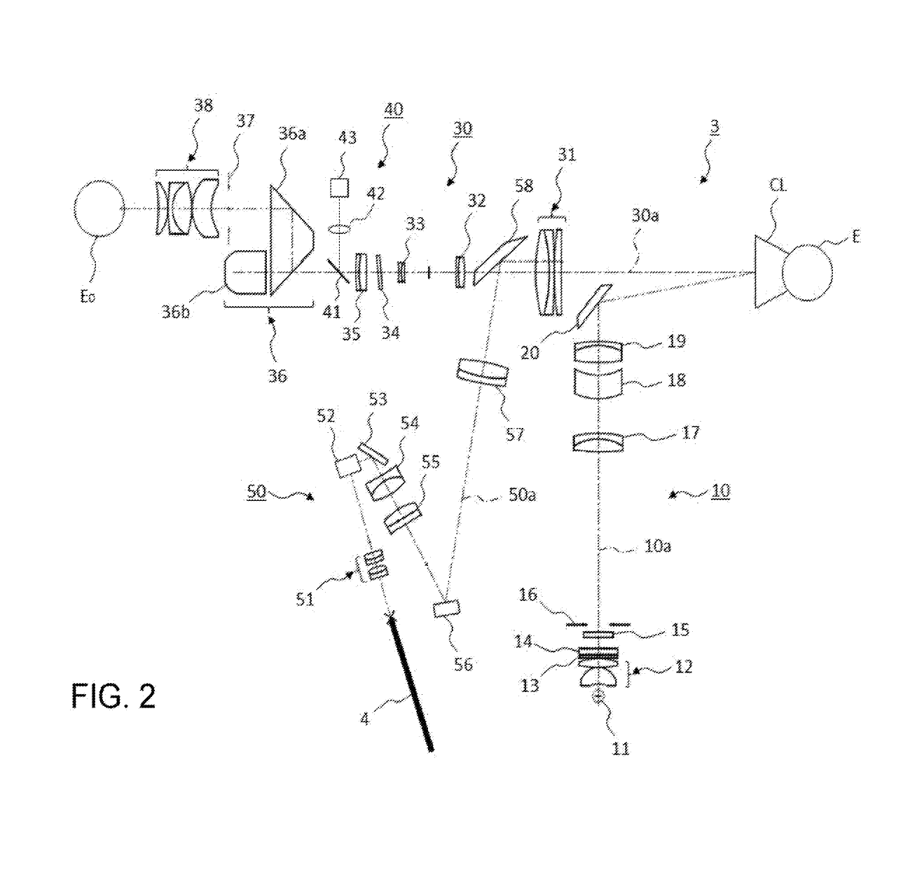

The slit lamp microscope 3 includes an illumination part 3a, an observation part 3b, an eyepiece part 3c, and a laser irradiation part 3d. The illumination part 3a accommodates an illumination system 10 illustrated in FIG. 2. The observation part 3b and the eyepiece part 3c accommodate an observation system 30. The observation part 3b further accommodates an imaging system 40. The laser irradiation part 3d accommodates a laser irradiation system 50.

Although not illustrated, as with conventional microscopes, the slit lamp microscope 3 has operation members such as a lever, a handle, a button, a knob, and the like. These operation members belong to the operation unit 6 from the functional point of view. In the configuration illustrated in FIG. 1, the processing unit 5 controls the slit lamp microscope 3 in response to a signal from the operation unit 6. However, in addition to or instead of the mechanism that operates by an electrical driving force like this, the slit lamp microscope 3 may have a mechanism that operates by a driving force applied by the operator.

### (Optical System of the Slit Lamp Microscope 3)

With reference to FIG. 2, the optical system of the slit lamp microscope 3 is described. In FIG. 2, a contact lens CL is directly applied to the patient's eye E for the laser treatment of the fundus Ef and the corner. The slit lamp microscope 3 includes the illumination system 10, the observation system 30, the imaging system 40, and the laser irradiation system 50.

### (Illumination System 10)

The illumination system 10 emits illumination light for observing the patient's eye E. The illumination part 3a is configured to be capable of changing the direction of an optical axis 10a (illumination optical axis) of the illumination system 10 in the lateral direction and the vertical direction. Thereby, it is possible to arbitrarily change the illumination direction for observation of the patient's eye E.

The illumination system 10 includes a light source 11, a condenser lens 12, filters 13, 14 and 15, a slit diaphragm 16, imaging lenses 17, 18 and 19, and a deflecting member 20.

The light source 11 outputs illumination light. Incidentally, the illumination system 10 may be provided with a plurality of light sources. For example, the light source 11 may include both of a light source for emitting fixed light (continuous light) (halogen lamp, LED, etc.) and a light source for emitting flash light (xenon lamp, LED, etc.). Besides, the illumination system 10 may include a light source for fundus observation and a light source for anterior eye observation separately. The light source 11 outputs at least visible light. Further, the light source 11 may be capable of outputting infrared light. The light source 11 may include both of a visible light source that outputs visible light and an infrared light source that outputs infrared light. The condenser lens 12 is a lens (or a lens system) for collecting light output from the light source 11. The processing unit 5 controls the operation of the light source 11.

The filters 13 to 15 are optical elements for removing or reducing certain components of the illumination light. Examples of the filters 13 to 15 include a blue filter, a red-free filter, a neutral density filter, a heat-insulating filter, a cornea fluorescence filter, a color temperature conversion filter, a color rendering property conversion filter, a UV cut filter, an infrared light cut filter, a visible light cut filter, and the like. Each of the filters 13 to 15 can be inserted in and removed from the optical path of the illumination light. The processing unit 5 controls the insertion and removal of the filters 13 to 15.

The slit diaphragm 16 forms a slit to generate slit light. The slit diaphragm 16 includes a pair of slitting blades. The slit width is changed by changing the spacing between the slitting blades. The direction of the slit is changed by integrally rotating the slitting blades. On that occasion, the illumination optical axis 10a serves as the rotation center. Incidentally, the illumination system 10 may include a diaphragm in addition to the slit diaphragm 16. Examples of such a diaphragm include an illumination diaphragm for changing the amount of illumination light, an illumination field diaphragm for changing the size of the illumination field, and the like. Further, it is possible to change the amount of illumination light and the size of the illumination field by using a member other than the diaphragms. As an example of such a member may be cited a liquid crystal shutter. The processing unit 5 controls the operations of the slit diaphragm 16, the illumination diaphragm, the illumination field diaphragm, and the liquid crystal shutter.

The imaging lenses 17, 18 and 19 constitute a lens system for forming an image of the illumination light. The deflecting member 20 deflects the illumination light having passed through the imaging lenses 17, 18 and 19 to irradiate the patient's eye E with the light. The deflecting member 20 may be, for example a reflecting mirror or a reflecting prism.

The illumination system 10 may include a member other than those described above. For example, a diffuser panel may be removably arranged in the downstream of the deflecting member 20. The diffuser panel diffuses the illumination light to uniformize the brightness of the illumination field. There may also be provided a background light source for illuminating the background region of the illumination field by the illumination light.

### (Observation System 30)

The observation system 30 is an optical system for guiding the illumination light returning from the patient's eye E to the operator's eye E₀. The observation system 30 includes a pair of right and left optical systems to enable observation with both right and left eyes. The right and left optical systems have substantially the same structure, and therefore only one of them is illustrated in FIG. 2.

The observation part 3b is configured to be capable of changing the direction of an optical axis 30a (observation optical axis) of the observation system 30 in the lateral direction and the vertical direction. Thereby, it is possible to arbitrarily change the observation direction of the patient's eye E.

The observation system 30 includes an objective lens 31, zoom lenses 32 and 33, a protection filter 34, an imaging lens 35, a deflector 36, a field diaphragm 37, and an ocular lens 38.

The objective lens 31 is arranged in a position facing the patient's eye E. The zoom lenses 32 and 33 constitute a variable magnification optical system (zoom lens system). Each of the zoom lenses 32 and 33 is movable along the observation optical axis 30a. As another example of the variable magnification optical system, the observation system 30 may include a plurality of zoom lens groups, which can be selectively inserted in the optical path thereof. These zoom lens groups are configured to provide different magnifications. A zoom lens group arranged in the optical path of the observation system 30 is used as the zoom lenses 32 and 33. This variable magnification optical system allows a change in the magnification (angle of view) of an image of the patient's eye E observed through the ocular lens 38 and observed as a captured image. For example, the magnification is changed by using an observation magnification operation knob included in the operation unit 6. Besides, the processing unit 5 may control the magnification based on the operation on a switch or the like included in the operation unit 6.

The protection filter 34 is configured to shield the treatment light LT to thereby protect the operator's eye E₀ from the laser beam. For example, the protection filter 34 is inserted into the optical path in response to a trigger to start a laser treatment (or laser output). During normal observation, the protection filter 34 is removed from the optical path. The processing unit 5 controls the insertion and removal of the protective filter 34. A multi-layer structured filter may be used to reduce changes in apparent color. This filter is kept located in the optical path, for example.

The imaging lens 35 is a lens (or a lens system) for forming an image of the patient's eye E. The deflector 36 is an optical member that moves the traveling direction of light in parallel to match it with the eye width of the operator, and includes prisms 36a and 36b. The ocular lens 38 moves integrally with the deflector 36. The deflector 36 and the ocular lens 38 are accommodated in the eyepiece part 3c. Other members of the observation system 30 are accommodated in the observation part 3b.

### (Imaging System 40)

The imaging system 40 is configured to capture an image of the patient's eye E. The imaging system 40 includes a beam splitter 41, an imaging lens 42, and an image sensor 43. The imaging system 40 is branched from the observation system 30. The beam splitter 41 is located between the imaging lens 35 and the deflector 36 of the observation system 30. The beam splitter 41 may be a half mirror, for example. The imaging lens 42 is a lens (or a lens system) for forming an image of the patient's eye E on the image sensor 43. The image sensor 43 may be an area sensor including am imaging device such as CCD and CMOS, for example. Signals (image signals, video signals) output from the image sensor 43 are sent to the processing unit 5.

The observation system 30 may be provided with the imaging system 40 for either one or both the right and left optical systems. If both the right and left optical systems are provided with the imaging system 40, a stereoscopic image of the patient's eye E can be obtained. The stereoscopic image is a still image or a moving image. The stereoscopic image is used for stereoscopic observation (described later) or the like.

### (Laser Irradiation System 50)

The laser irradiation system 50 is configured to guide the irradiation light, which has been transmitted from the light source unit 2 to the slit lamp microscope 3 via the optical fiber 4, to the patient's eye E. The laser irradiation system 50 includes a collimator lens 51, an optical scanner 52, a mirror 53, relay lenses 54 and 55, a mirror 56, a collimator lens 57, and a deflecting member 58.

The collimator lens 51 collimates the irradiation light output from the optical fiber 4 into a parallel light flux. The optical scanner 52 two-dimensionally deflects the irradiation light. The optical scanner includes a pair of galvanometer scanners, for example. The processing unit 5 controls the operation of the optical scanner 52.

The mirror 53 reflects the irradiation light having passed through the optical scanner 52 to change the traveling direction. The relay lenses 54 and 55 relay the irradiation light having reflected by the mirror 53. The mirror 56 reflects the irradiation light having passed through the relay lenses 54 and 55 to change the traveling direction. The collimator lens 57 collimates the irradiation light having passed through the relay lenses 54 and 55 into a parallel light flux. The deflecting member 58 is located behind the objective lens 31 and deflects the irradiation light having passed through the collimator lens 57, thereby projecting the patient's eye E with the irradiation light.

### (Contact Lens CL)

When laser treatment is applied to the fundus Ef, the corner, or the like, the contact lens CL is in contact with the cornea Ec. A plurality of contact lenses with different magnifications, shapes, or the like is prepared to perform a variety of laser treatments. This allows the user to select a contact lens depending on the treatment type, the treatment site, the conditions of the patient's eye E, or the like.

### (Irradiation Conditions)

In this example, irradiation light in a predetermined pattern is applied to the patient's eye E. There are various conditions (irradiation conditions) in the pattern of the irradiation light. A projection image of irradiation light is called a spot. The irradiation conditions include the array pattern of a plurality of spots (array condition), the size of the array pattern (array size condition), the orientation of the array pattern (array direction condition), the size of each spot (spot size condition), interval between the spots (spot interval condition), and the like. The irradiation conditions further include those related to items other than the pattern and the spot. For example, the irradiation conditions include the intensity (power) and the wavelength of the irradiation light. The control based on the irradiation conditions is performed in the same manner as in the conventional laser treatment apparatus of the pattern-irradiation type.

### (Control System)

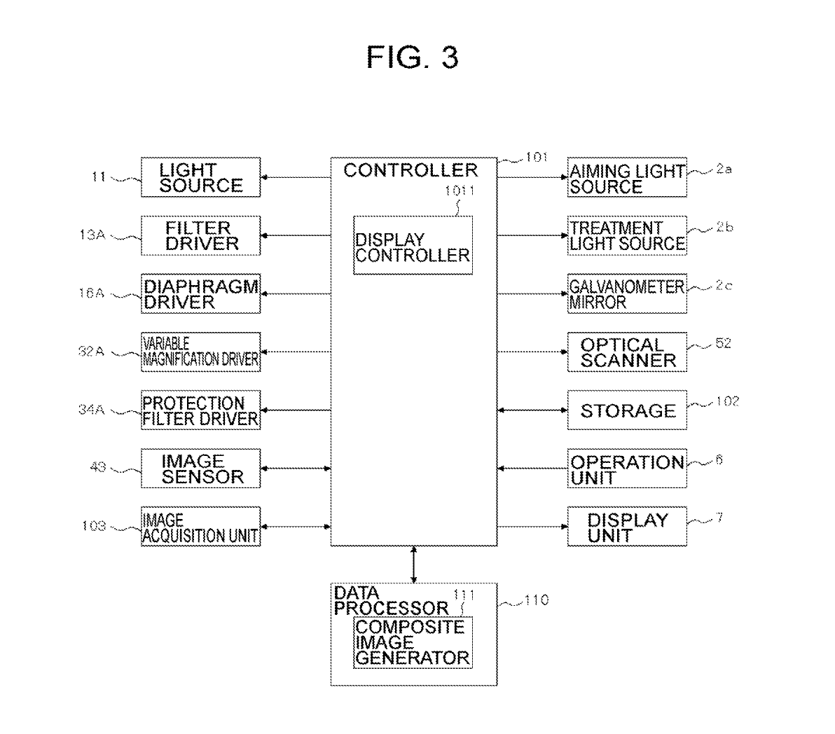

The control system of the laser treatment apparatus 1 is described with reference to FIG. 3. A controller 101 of the processing unit 5 serves as the center of the control system of the laser treatment apparatus 1. In FIG. 3, illustration of some parts is omitted.

### (Controller 101)

The controller 101 controls each unit of the laser treatment apparatus 1. For example, the controller 101 controls the light source unit 2, the display unit 7, the illumination system 10, the observation system 30, and the laser irradiation system 50. Besides, the controller 101 controls the movement of the contact lens CL. The controller 101 controls at least constituent parts illustrated in FIG. 3. For example, the controller 101 performs the control based on the irradiation conditions described above.

As the control of the light source unit 2, the controller 101 controls the aiming light source 2a, the treatment light source 2b, the galvanometer mirror 2c, and the like. The control of the aiming light source 2a and the treatment light source 2b includes the ON/OFF control of the output of the irradiation light, the control of the output intensity (output light amount) of the irradiation light, and the like. Besides, if there are provided one or more treatment light sources (2b) capable of outputting different types of treatment light LT, the controller 101 controls the treatment light source(s) to selectively output the treatment light LT. The control of the galvanometer mirror 2c includes a control for changing the orientation of the reflecting surface of the galvanometer mirror 2c.

The display unit 7 displays various kinds of information under the control of the controller 101. The display unit 7 includes an arbitrary display device such as a flat panel display (LCD, etc.), a CRT display, or the like. For example, the slit lamp microscope 3 or the processing unit 5 (computer) is provided with the display unit 7. The display controller 1011 controls the display unit 7.

As the control of the illumination system 10, the controller 101 controls the light source 11, the filters 13 to 15, the slit diaphragm 16, and other diaphragm members. The control of the light source 11 includes the ON/OFF control of the output of the illumination light, the control of the output intensity (output light amount) of the illumination light, and the like. The control of the filters 13 to 15 includes a control for inserting and removing each of the filters 13 to 15 with respect to the illumination optical axis 10a. The control of the filters 13 to 15 is performed by controlling a filter driver 13A. The control of the slit diaphragm 16 includes a control for changing the spacing between the slitting blades and a control for integrally moving/rotating the slitting blades. The former control corresponds to controlling the change of the slit width. The latter control corresponds to controlling the change of a position to emit the illumination light (slit light) while the slit width is maintained constant. As described above, the other diaphragm members include an illumination diaphragm for changing the amount of illumination light and an illumination field diaphragm for changing the size of the illumination field. The slit diaphragm 16, the illumination diaphragm, and the illumination field diaphragm are individually controlled by controlling a diaphragm driver 16A.

As the control of the observation system 30, the controller 101 controls the zoom lenses 32 and 33, the protection filter 34, and the like. As the control of the zoom lenses 32 and 33, the controller 101 controls a variable magnification driver 32A to move them along the observation optical axis 30a or to arrange a zoom lens group with different magnification in the optical path of the observation system 30. Thereby, the observation magnification (angle of view) is changed. As the control of the protection filter 34, the controller 101 controls a protection filter driver 34A to insert or remove the protective filter 34 with respect to the observation optical axis 30a.

As the control of the laser irradiation system 50, the controller 101 controls the optical scanner 52 and the like. For example, the controller 101 changes the orientation of two galvanometer mirrors of the optical scanner 52. Thereby, the irradiation light incident from the light source unit 2 via the optical fiber 4 can be two-dimensionally deflected.

The controller 101 also controls the process of reading data from a storage 102 and writing data to the storage 102.

The controller 101 includes a microprocessor, a RAM, a ROM, a hard disk drive, and the like. The hard disk drive stores computer programs such as a control program in advance. The operation of the controller 101 is realized by the cooperation of the computer programs and the above-mentioned hardware. The controller 101 may include a communication device for communicating with an external device.

### (Storage 102)

The storage 102 stores various kinds of data and computer programs. The storage 102 includes storage devices such as a RAM, a ROM, and a hard disk drive, for example.

### (Image Acquisition Unit 103)

An image acquisition unit 103 acquires an image of the patient's eye E. The image acquired by the image acquisition unit 103 is a wide-area image that represents a region of the patient's eye E including the illumination field of slit light from the illumination system 10. The wide-area image represents a wider area as compared to the image captured by using slit light (slit photography image). For example, the wide-area image represents an area as wide as or wider than the imaging field (illumination field) when the slit diaphragm 16 is fully opened.

In one example, the image acquisition unit 103 may have a structure to receive a wide-area image stored outside the laser treatment apparatus 1. This structure includes, for example, a communication device for sending/receiving data to/from an external device via a network, and/or a device (drive device, etc.) for reading data stored in a recording medium.

In another example, the image acquisition unit 103 may have a structure to capture images of the patient's eye E. Specifically, the image acquisition unit 103 may be part of the slit lamp microscope 3 or include at least part of another modality. Examples of the other modality include imaging apparatuses such as a fundus camera, a scanning laser ophthalmoscope (SLO), an optical coherence tomography (OCT), an ultrasonic diagnosis apparatus, and a magnetic resonance imaging (MRI) apparatus.

Examples of the wide-area image include an infrared image captured by using infrared light, a color image captured by using visible light, an image captured by using visible light from which a predetermined component has been removed (red-free image, etc.), a fluorescent contrast image captured by having administered a fluorescent agent to the patient, an autofluorescence contrast image captured without having administered a fluorescent agent, a fundus image captured by using a fundus camera, an image (an anterior segment image, a fundus image, a corner image, etc.) captured by using a slit lamp microscope, an SLO image captured by SLO, an OCT image captured by using an OCT apparatus, an ultrasound image captured by using an ultrasound diagnosis apparatus, an MRI image captured by using an MRI apparatus, and the like.

The wide-area image may be a map (distribution map) that represents distribution of a predetermined parameter obtained by analyzing an image of the patient's eye E. For example, the wide-area image may be a distribution map of the retinal thickness obtained by analyzing a three-dimensional image captured by using OCT. Alternatively, the wide-area image may be a distribution map of predetermined parameters obtained by examining the patient's eye E. For example, the wide-area image may be a distribution map of the visual field and/or the sensitivity obtained by performing the visual field test (perimetry).

### (Data Processor 110)

The data processor 110 performs various kinds of data processing. The data processor 110 includes a composite image generator 111.

### (Composite Image Generator 111)

The composite image generator 111 generates a composite image of a wide-area image acquired by the image acquisition unit 103 and a slit photography image based on the output of the image sensor 43. The composite image generator 111 performs at least registration between the wide-area image and the slit photography image.

Described below is an example of the registration process. The composite image generator 111 analyzes the slit photography image to specify a first image area corresponding to a predetermined site of the patient's eye E. Similarly, the composite image generator 111 analyzes the wide-area image to specify a second image area corresponding to the predetermined site of the patient's eye E. These analyses involve known image processing for specifying an area in an image. Then, the composite image generator 111 adjusts the positions of the slit photography image and the wide-area image so that the first image area and the second image area match with each other. Here, considering that the area represented by the wide-area image includes the area represented by the slit photography image, the composite image generator 111 may calculate the coordinates (area) of the slit photography image in the coordinate system of the wide-area image.

Besides, the composite image generator 111 may combine the wide-area image and the slit photography image into a single image. Such processing may be implemented by embedding the slit photography image in the wide-area image after the position registration described above.

If the images are not combined into a single image, the slit photography image and the wide-area image after the position registration may be displayed as being superimposed one on top of the other by using, for example, the layer function. The superimposed image thus obtained is an example of the composite image.

When the slit photography image and/or the wide-area image are/is moving image(s), the position registration is performed with respect to each frame. The same applies to the process of combining the images to generate a single image as well as the process of displaying the images superimposed one upon the other.

### (Operation Unit 6, Display Unit 7)

The operation unit 6 includes a variety of hardware keys and/or software keys. The display unit 7 includes, for example, a flat panel display. At least part of the operation unit 6 and at least part of the display unit 7 may be formed integrally. One example of this is a touch panel display.

### [Display of Composite Image]

A display controller 1011 displays the composite image generated by the composite image generator 111 on the display unit 7. When the composite image generator 111 generates a single image as described above, the display controller 1011 displays the single image on the display unit 7. On the other hand, if the composite image generator 111 does not generate a single image (e.g., when only performing position registration), the display controller 1011 displays the wide-area image in the first layer and the slit photography image in the second layer. At this time, the second layer may be placed in front of the first layer. In addition, the opacity (alpha value) of the first layer and/or the second layer can be arbitrarily set.

When the slit photography image and/or the wide-area image are/is moving image(s), the composite image generator 111 generates a series of composite images (frames). The display controller 1011 displays the series of composite images on the display unit 7 as a moving image. This moving image display process is accomplished by displaying the series of composite images, sequentially in chronological order, at a predetermined frame rate. The moving image display process is described in more detail in the second embodiment.

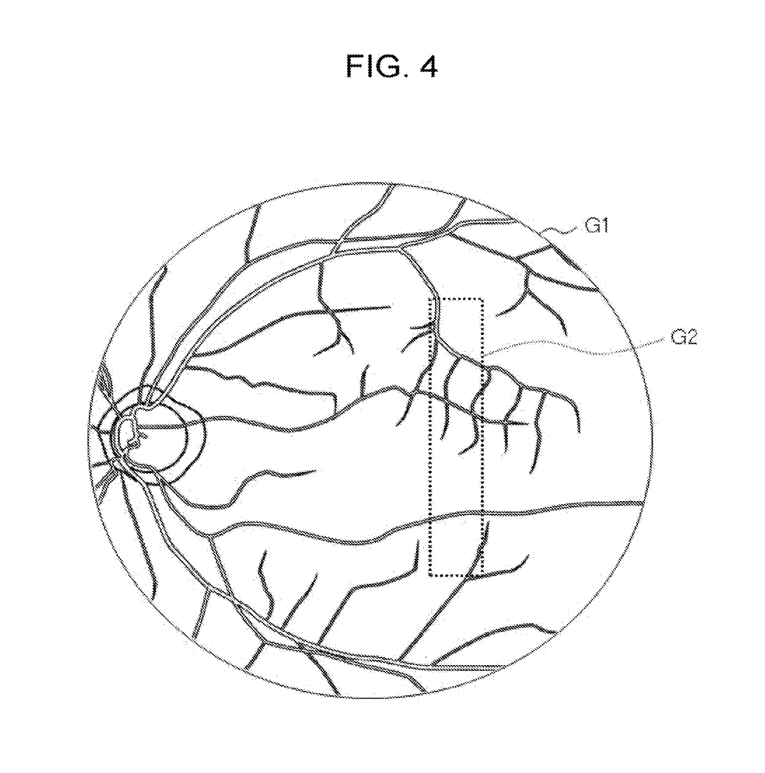

FIG. 4 is a schematic diagram illustrating an example of a display mode of a composite image. FIG. 4 illustrates a composite image displayed when laser treatment is applied to the fundus Ef. The composite image is formed of a wide-area image G1 and a slit photography image G2. The slit photography image G2 is a front image that represents the morphology of the fundus Ef in the irradiation area of slit light. The wide-area image G1 is a front image that represents the morphology of the fundus Ef in a region including the area of the slit photography image G2.

### [Effects]

Described below are the actions and effects of the laser treatment apparatus 1 according to the first embodiment.

According to the first embodiment, the laser treatment apparatus (1) is used to apply laser treatment to a patient's eye (E). The laser treatment apparatus (1) includes an irradiation system (the light source unit 2, the laser irradiation system 50, etc.), an illumination system (10), an imaging system (the observation system 30, the imaging system 40), an image acquisition unit (103), and a composite image presenting unit (the composite image generator 111, the display controller 1011, etc.).

The irradiation system irradiates the patient's eye with laser beams emitted from a light source (the aiming light source 2a, the treatment light source 2b). The illumination system illuminates the patient's eye with slit light. The imaging system guides the slit light returning from the patient's eye to an imaging device (the image sensor 43). The image acquisition unit acquires a first image (wide-area image) that represents a region of the patient's eye including the illumination field of the slit light. The composite image presenting unit presents a composite image of the first image (wide-area image) acquired by the image acquisition unit and a second image (slit photography image) of the patient's eye based on the output of the imaging device.

According to the embodiment thus configured, while observing a relatively narrow area to be aimed at or to be irradiated with laser beams by using slit light, the operator can check, with a wide-area image, the state of a relatively wide area that includes the narrow area. Thus, the operator can easily figure out the site of the patient's eye which is to be aimed at or to be irradiated with laser beams, while controlling the irradiation range of slit light not to dazzle the patient.

### Second Embodiment

The second embodiment describes typical examples of the case where the slit photography image is a moving image. A laser treatment apparatus of the present embodiment may have an overall configuration and optical systems basically the same as described in the first embodiment (see FIGS. 1 and 2). Components and reference numerals in the first embodiment are applied as appropriate.

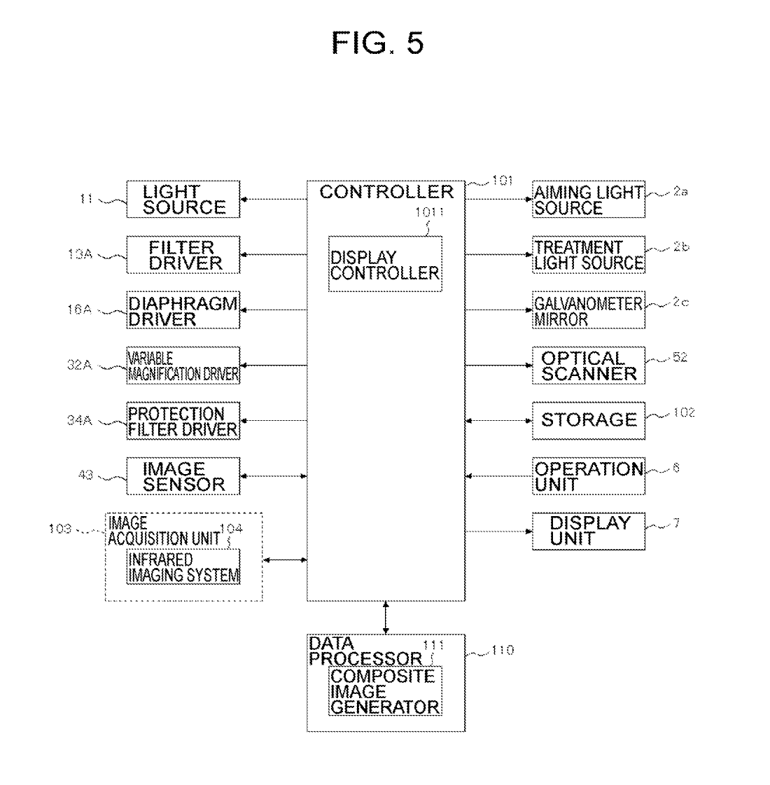

FIG. 5 illustrates an example of the configuration of the control system of the laser treatment apparatus. The image acquisition unit 103 of the present embodiment includes an infrared imaging system 104. The infrared imaging system 104 captures an image of the patient's eye E using infrared light. In particular, the infrared imaging system 104 of the present example is capable of capturing a moving image using infrared light. The infrared imaging system 104 is provided separately from the imaging system 40 for acquiring a slit photography image (but the imaging systems 104 and 40 may share a part in common). Accordingly, in the present example, imaging with slit light (visible light) and imaging with infrared light can be performed in parallel with each other.

The infrared imaging system 104 includes, for example: an optical system for illuminating the patient's eye E with infrared light output from the infrared light source; and an optical system for guiding the infrared light returning from the patient's eye E to the imaging device having sensitivity in the infrared region. The optical path of the infrared imaging system 104 and that of the imaging system 40 may have a common part. In this case, the optical path of the infrared imaging system 104 and that of the imaging system 40 are separated from each other by means of a beam splitter such as a dichroic mirror.

Besides, the infrared imaging system 104 and the imaging system 40 may be arranged coaxially. This facilitates the registration between images acquired by the both.

The infrared imaging system 104 and the imaging system 40 may have a common zoom system (the zoom lenses 32 and 33) or separate zoom systems. When the imaging systems 104 and 40 share the same zoom system, the configuration of the optical system can be simplified. In addition, this facilitates the registration between images. If having their individual zoom systems, the infrared imaging system 104 and the imaging system 40 can each capture an image of an arbitrary magnification (angle of view). The registration between images can be achieved by taking into account the magnifications of the both, or performing image processing.

A description is given of typical examples of the application of the present embodiment. The following examples may be combined arbitrarily.

Described below is the first application example. In the present embodiment, a slit photography image (second image) is a moving image. The composite image generator 111 generates a composite image of a wide-area image and each frame of a slit photography image. The image composition processing may be performed in the same manner as in the first embodiment. If the wide-area image is a still image, each frame of the slit photography image is combined with the still image. Another example is described later in which the wide-area image is also a moving image.

The display controller 1011 displays the composite image thus generated. At this time, the display controller 1011 can display a moving image based on a plurality of composite images (composite frames) corresponding to the frames of the slit photography image. When the wide-area image is a still image, the slit photography image is displayed as a moving image with the same wide-area image as the background. During the display of the moving image, the wide-area image can be changed to another one (still image, etc.). Note that the above processing is not necessarily performed on all the frames of the slit photography image (moving image), and frames may be thinned out at a predetermined rate.

Described below is the second application example. A composite image can be presented in parallel with the acquisition of a slit photography image (capturing of a moving image with slit light). That is, it is possible to present a composite image of a wide-area image and a slit photography image in real time while a moving image of the patient's eye E is being captured with slit light. The following is an example of the operation of the laser treatment apparatus to implement this.

The image sensor 43 is used to capture an image by using slit light. The image sensor 43 captures images at a predetermined imaging rate. The image sensor 43 sequentially outputs image signals each corresponding to a frame at a timing synchronous with the imaging rate. The composite image generator 111 sequentially receives the image signals output from the image sensor 43 in real time. The composite image generator 111 combines each of the frames received sequentially in real time with a wide-area image. The display controller 1011 sequentially receives composite images (composite frames) thus generated in real time. The display controller 1011 displays a moving image based on the composite frames received in real time at a frame rate in synchronization with the imaging rate, for example.

Described below is the third application example. In this example, both the slit photography image and the wide-area image are moving images. The slit light is visible, and the slit photography image is a visible moving image. The wide-area image is an infrared moving image captured by the infrared imaging system 104. The slit photography image can be acquired in parallel with the acquisition of the wide-area image (i.e., the photographing of them can be performed in parallel). Further, as in the second application example, a composite image can be presented in parallel with at least one of the acquisition of the visible image and that of the infrared image (i.e., the composite image can be displayed in real time).

The composite image generator 111 is fed with frames of the slit photography image as well as frames of the wide-area image. When presenting composite images in real time, the composite image generator 111 is sequentially fed with frames of the slit photography image in real time while being sequentially fed with frames of the wide-area image in real time.

The composite image generator 111 associates the frame of the slit photography image with the frame of the wide-area image. When receiving both the frames in real time, the composite image generator 111 may perform frame association based on input timings of the frames. In addition, when the imaging system 40 and the infrared imaging system 104 perform imaging in synchronization with each other, the frames are associated based on the synchronization control. For another example, the frames are associated by using biological information. For example, the frames can be associated based on the heart rate of the patient (electrocardiogram, blood flow information obtained by OCT, etc.).

Next, the composite image generator 111 generates a composite image (composite frame) of the frame of the slit photography image and the frame of the wide-area image associated with each other. For example, the display controller 1011 sequentially receives composite frames thus generated in real time. The display controller 1011 displays a moving image based on the composite frames sequentially received from the composite image generator 111 at a frame rate in synchronization with the imaging rate of the slit photography image and/or the wide-area image, for example.

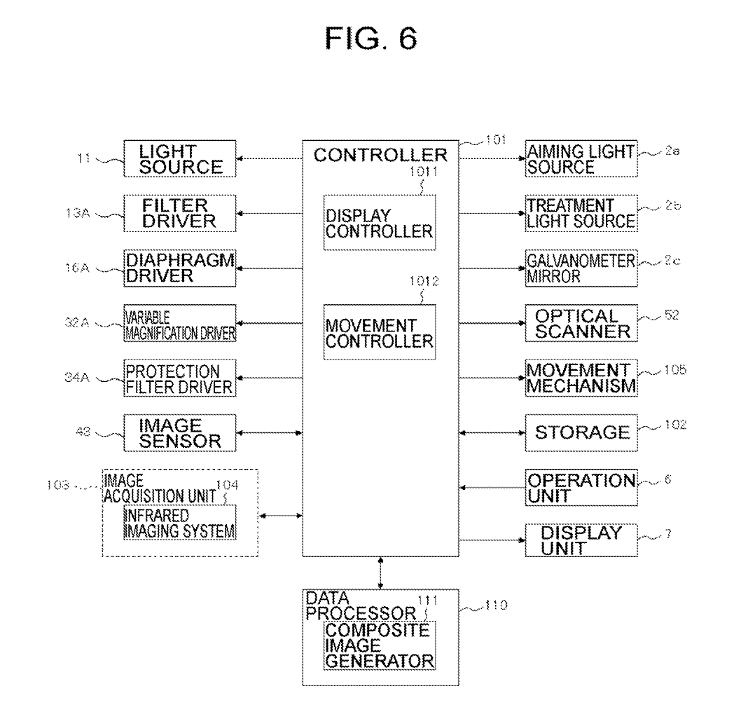

In the following, a modification of the second embodiment is described. FIG. 6 illustrates an example of the configuration of the control system of the present modification. As illustrated in FIG. 6, the laser treatment apparatus further includes a movement mechanism 105, and the controller 101 includes a movement controller 1012.

The movement mechanism 105 has a function of moving the irradiation system (at least part of the laser irradiation system 50), the illumination system (at least part of the illumination system 10), and the imaging system (at least part of the observation system 30 and the imaging system 40). The movement mechanism 105 includes, for example, a movable stage and an actuator configured to move the movable stage. On the movable stage, the optical system and the like to be moved are mounted.

The movement controller 1012 controls the movement mechanism 105 based on an infrared moving image captured by the infrared imaging system 104, thereby causing the irradiation system, the illumination system, and the imaging system to follow the movement of the patient's eye E. In other words, the movement controller 1012 performs tracking of the irradiation system etc. with respect to the patient's eye E. The tracking is implemented by, for example, performing a series of processes as follows in real time: (1) a process of sequentially specifying an image area corresponding to a predetermined site of the patient's eye E from frames of an infrared moving image captured in real time; (2) a process of obtaining a temporal change in the position of the image area sequentially specified (i.e., the displacement of the image area among frames); and (3) a process of controlling the movement mechanism 105 to cancel the displacement of the image area sequentially obtained. Incidentally, the data processor 110 may be configured to perform the processes (1) and (2). Examples of the predetermined site in the process (1) include optic disc, blood vessel, lesion, corner, pupil, and the like.

Described below are the actions and effects of the laser treatment apparatus according to the second embodiment.

According to the second embodiment, the imaging device (the image sensor 43) captures a moving image of a patient's eye (E). The second image (slit photography image) includes the moving image thus captured. The composite image presenting unit (the composite image generator 111 and the display controller 1011) presents a composite image of the moving image and the first image (wide-area image).

With this configuration, while observing a relatively narrow area to be aimed at or to be irradiated with laser beams on a moving image using slit light, the operator can check the state of a relatively wide area that includes the narrow area on a wide-area image.

According to the present embodiment, the composite image presenting unit can present the composite image in parallel with the acquisition of the moving image using slit light. That is, while a moving image of the patient's eye E is being captured with slit light, a composite image of a slit photography image (moving image) and a wide-area image can be presented in real time.

With this configuration, while viewing a relatively narrow area to be aimed at or to be irradiated with laser beams on a moving image using slit light, the operator can check the state of a relatively wide area that includes the narrow area on a wide-area image.

In the present embodiment, the slit light may include visible light, and the second image may be a visible moving image. Besides, the image acquisition unit (103) may include an infrared imaging system (104) configured to capture a moving image of the patient's eye using infrared light. Further, the first image (wide-area image) may include an infrared moving image captured by the infrared imaging system. The composite image presenting unit is capable of presenting a composite image of the infrared moving image and the visible moving image.

With this configuration, while viewing a relatively narrow area to be aimed at or to be irradiated with laser beams on a moving image using slit light (visible light), the operator can check the state of a relatively wide area that includes the narrow area on a moving wide-area image using infrared light. Since the wide-area image is the infrared moving image, the patient cannot view it and views only the moving image using slit light as in the case of the conventional apparatus. Thus, the operator can check the state of a wider area while the patient feels the same level of dazzling as in the conventional apparatus.

In the present embodiment, the visible moving image can be captured by the imaging device in parallel with the capturing of the infrared moving image by the infrared imaging system. Further, the composite image presenting unit is capable of presenting the composite image in parallel with the acquisition of the visible moving image and the acquisition of the infrared moving image.

With this configuration, while observing a relatively narrow area to be aimed at or to be irradiated with laser beams in real time on a moving image using slit light (visible light), the operator can observe the state of a relatively wide area that includes the narrow area in real time on a moving wide-area image using infrared light.

The laser treatment apparatus of the present embodiment may further include: a movement mechanism (105) configured to move the irradiation system, the illumination system, and the imaging system; and a movement controller (1012) configured to control the movement mechanism based on an infrared moving image captured by the infrared imaging system, thereby causing the irradiation system, the illumination system, and the imaging system to follow the movement of the patient's eye.

With this configuration, in any of the observation modes of the present embodiment described above, the optical system and the like can be made to track the patient's eye. Thus, it is possible to reduce the influence of the movement of the patient's eye (eyeball movement, pulsation, body motion, etc.) on the observation, photographing, aiming, laser irradiation, and the like.

### Third Embodiment

The third embodiment describes typical examples of the case where information is presented together with a composite image of a slit photography image and a wide-area image. A laser treatment apparatus of the present embodiment may have an overall configuration and optical systems basically the same as described in the first embodiment (see FIGS. 1 and 2). Components and reference numerals in the first embodiment are applied as appropriate.

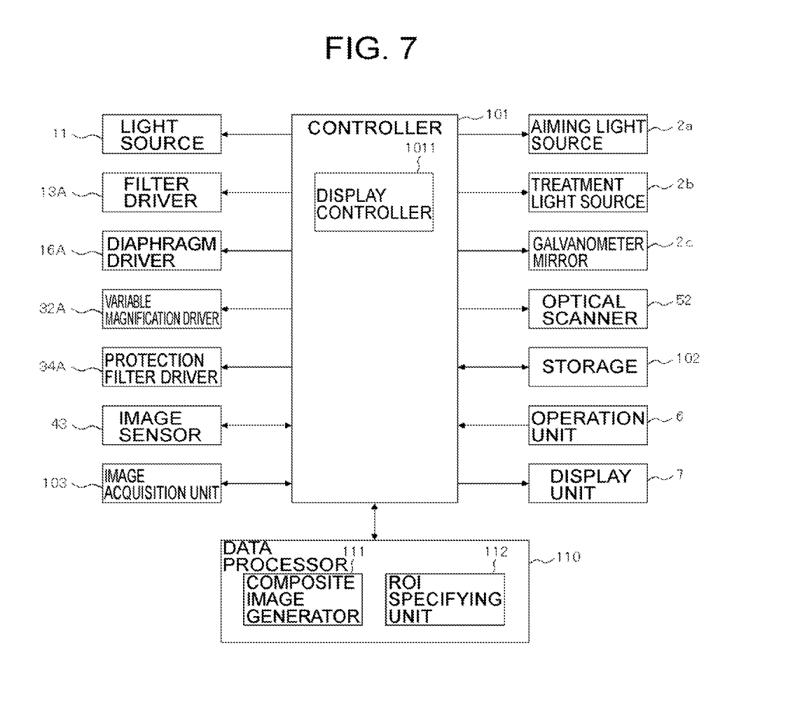

FIG. 7 illustrates an example of the configuration of the control system of the laser treatment apparatus. The data processor 110 of the present example further includes a region of interest (ROI) specifying unit 112. The ROI specifying unit 112 functions as a part of the composite image presenting unit, and analyzes a wide-area image (first image) to thereby specify a region of interest corresponding to a site of interest of the patient's eye. Examples of the site of interest of the patient's eye E include optic disc, macula, blood vessel, corner, lesion, area to be treated with laser therapy, and the like. The process of specifying a region of interest in a wide-area image involves image processing such as, for example, a known region specifying process related to the pixel value (threshold processing, binarization, contour extraction, image filtering, etc.), pattern matching, and the like. The area to be treated with laser therapy, which is an example of the site of interest, is set in, for example, the preoperative planning conducted in preparation for the laser treatment.

The first image (wide-area image) may be a fluorescent contrast image. The fluorescent contrast image is used to specify the location of a blood vessel, the range of bleeding, and the like. The ROI specifying unit 112 analyzes a fluorescent contrast image to specify the region of interest corresponding to a lesion regarding the blood vessel, blood, or the like. This process of ROI specification includes, for example, a known region specifying process related to the pixel value (threshold processing, binarization, contour extraction, image filtering, etc.).

The display controller 1011 presents information (supplementary information) indicating the region of interest specified by the ROI specifying unit 112 together with a composite image of a slit photography image and a wide-area image. The supplementary information may be embedded in the composite image. In this case, the composite image generator 111 embeds the supplementary information in the composite image. Alternatively, the composite image and the supplementary information may be displayed on different layers.

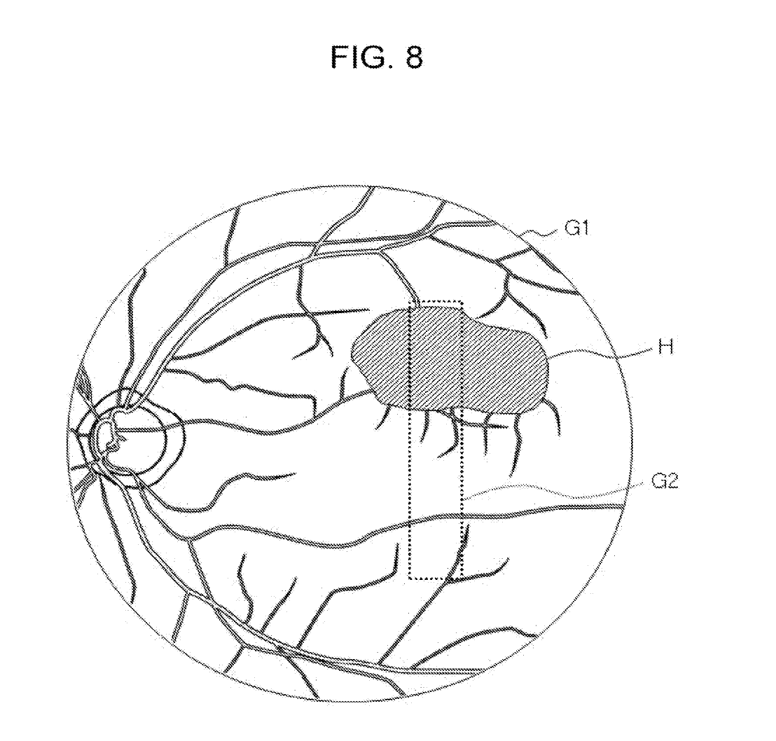

FIG. 8 illustrates an example of a display mode of the supplementary information. FIG. 8 illustrates the composite image of a wide-area image G1 and a slit photography image G2 (see FIG. 4). In addition, supplementary information H is displayed together with the composite image. The supplementary information H indicates, for example, an area to be treated with laser therapy set in the preoperative planning.

With the presentation of such supplementary information, the operator can be notified of the site of interest in the laser therapy. The site of interest may be a site to be treated with or not to be treated with laser therapy.

With reference to an example illustrated in FIG. 8, the operator can figure out the entire area to be treated with laser therapy by the wide-area image G1. The slit photography image G2 illustrates only a part of the area to be treated. With the conventional technology in which observation is performed by using only the slit photography image G2, the entire area to be treated with laser therapy cannot be recognized. On the other hand, according to the present embodiment, the entire area to be treated with laser therapy can be recognized. Therefore, the operator can easily recognize which part of the area to be treated is represented by the current slit photography image G2, which part of the area to be treated has already been performed aiming and/or laser irradiation, and the like.

### Fourth Embodiment

The fourth embodiment describes typical examples for presenting a composite image of a slit photography image and a wide-area image in the observation visual field. A laser treatment apparatus of the present embodiment may have an overall configuration as described in the first embodiment (see FIG. 1). Components and reference numerals in the first embodiment are applied as appropriate.

As described above, the observation system 30 guides the slit light returning from the patient's eye E to the ocular lens 38. Further, the observation system 30 includes a pair of right and left optical systems for observing the patient's eye E with both the eyes. That is, the observation system 30 is a binocular observation system including a pair of right and left ocular lenses 38.

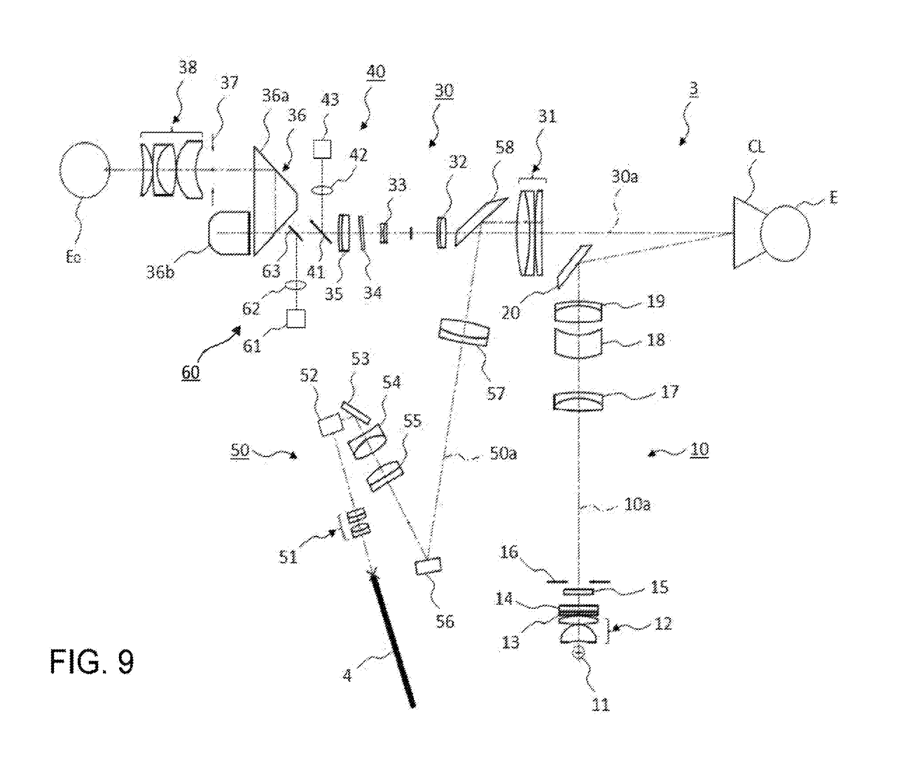

The composite image presenting unit of the present embodiment includes a presentation optical system for guiding a wide-area image (first image) or a composite image of the wide-area image and a slit photography image to the ocular lens(es) 38 through the optical path(s) of the observation system 30. FIG. 9 illustrates an example of the presentation optical system.

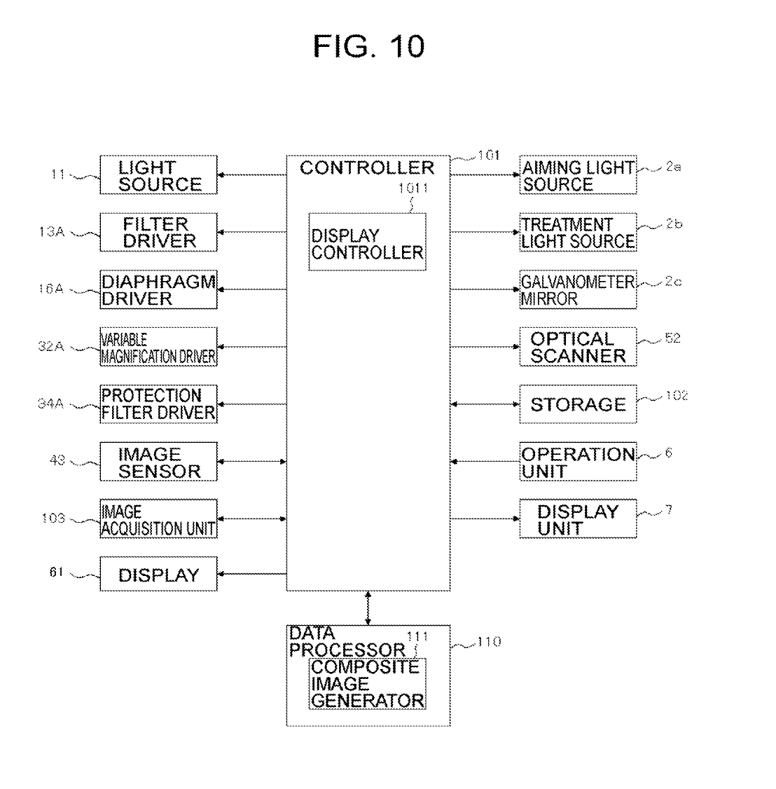

As illustrated in FIG. 9, a presentation optical system 60 includes a display 61, a condenser lens 62, and a beam splitter 63. The presentation optical system 60 is branched from the observation system 30. The display 61 includes, for example, a flat panel display or a micro projector. The display 61 operates under the control of the display controller 1011 (see FIG. 10). The beam splitter 63 is located, for example, between the beam splitter 41 and the deflector 36. The beam splitter 63 may be a half mirror, for example.

The display controller 1011 displays a wide-area image acquired by the image acquisition unit 103 or a composite image generated by the composite image generator 111 on the display 61. A light flux output from the display 61 passes through the condenser lens 62, and is reflected by the beam splitter 63. Thereby, the light flux is guided to the optical path of the observation system 30. Having entered the optical path of the observation system 30, the light flux passes through the deflector 36 and the field diaphragm 37 to be guided to the ocular lens 38.

The operator can view the wide-area image or the composite image through the ocular lens 38. That is, the operator can perform the microscopic observation of the patient's eye E using slit light in parallel with the observation of the wide-area image or the composite image presented by the presentation optical system 60. When the wide-area image is presented, the operator can view a superimposed image (composite image) of a slit photography image (microscopic observation image) and a wide-area image. When the composite image is presented, the operator can view a superimposed image (composite image) of a slit photography image and the composite image.

The display controller 1011 can adjust the position of the wide-area image or the composite image with respect to the slit photography image (microscopic observation image) of the patient's eye E. This process can be carried out in the same manner as the position registration between the slit photography image and the wide-area image as described in the first embodiment.

As described above, the observation system 30 is a binocular observation system. When the wide-area image acquired by the image acquisition unit 103 is a stereoscopic image, a left wide-area image to be presented for the left eye can be presented through the left optical system, while a right wide-area image to be presented for the right eye can be presented through the right optical system. As a specific example, the right and left optical systems may be each provided with the presentation optical system 60. With this configuration, the wide-area image as a stereoscopic image (or a composite image, in which at least the wide-area image is a stereoscopic image) can be presented to the operator.

As described in the first embodiment, both the right and left optical systems of the observation system 30 can be provided with the imaging system 40. In such a case that a pair of right and left imaging systems 40 is provided, a composite image of a wide-area image and a slit photography image acquired by the left imaging system can be presented through the left optical system, while a composite image of a wide-area image and a slit photography image acquired by the right imaging system can be presented through the right optical system. With this configuration, a composite image, in which at least the slit photography image is a stereoscopic image, can be presented to the operator. Incidentally, when the wide-area image is also a stereoscopic image, a composite image (stereoscopic composite image) of the stereoscopic wide-area image and the stereoscopic slit photography image can be presented to the operator.

When the stereoscopic image is presented to the operator, the operator may wear a member for stereoscopic observation (glasses, etc.). Alternatively, a configuration may be applied in which a member for stereoscopic observation can be arranged in the observation system 30. In another example, there may be provided a three-dimensional display that does not require a member for stereoscopic observation. In any case, it is only required that the composite image presenting unit is configured to guide a wide-area image (first image) or a composite image to a pair of ocular lenses 38 to be stereoscopically viewable through a pair of optical paths of the binocular observation system 30.

Note that the composite image, which is stereoscopically viewable, can be displayed on the display unit 7. The processing for that can be performed in the same manner as described above.

It is practically convenient to be able to arbitrarily switch on/off the presentation of the wide-area image or the composite image through the observation system 30. The operator can provide an instruction for this by using the operation unit 6. According to the output of the operation unit 6, the composite image presenting unit (the display controller 1011) switches the state where the wide-area image (first image) or the composite image is guided to the ocular lens 38 (that is, the state where the wide-area image or the composite image is viewable by the operator's eye E₀) to the state where the wide-area image (first image) or the composite image is not guided to the ocular lens 38 (that is, the state where the wide-area image or the composite image is not viewable by the operator's eye E₀), or vice versa. With this configuration, when only the microscopic observation of the patient's eye E using slit light is desired, the presentation of the wide-area image or the composite image can be turned off. On the other hand, when the observation of the wide-area image or the composite image is desired, the presentation thereof can be turned on.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A laser treatment apparatus configured to apply laser treatment to a patient's eye, the laser treatment apparatus comprising:
an irradiation system configured to irradiate the patient's eye with laser beams emitted from a light source;
an illumination system configured to illuminate the patient's eye with slit light;
an imaging system configured to guide the slit light returning from the patient's eye to an imaging device;
an image acquisition unit configured to acquire a first image that represents a region of the patient's eye including an illumination field of the slit light; and
a composite image presenting unit configured to present a composite image of the first image and a second image of the patient's eye based on output of the imaging device.

2. The laser treatment apparatus of claim 1, wherein
the imaging device is configured to capture a moving image of the patient's eye,
the second image includes the moving image, and
the composite image presenting unit is configured to present the composite image of the first image and the moving image.

3. The laser treatment apparatus of claim 2, wherein the composite image presenting unit is configured to present the composite image in parallel with capturing of the moving image.

4. The laser treatment apparatus of claim 2, wherein
the slit light includes visible light,
the second image is a visible moving image,
the image acquisition unit includes an infrared imaging system configured to capture the moving image of the patient's eye using infrared light,
the first image includes an infrared moving image captured by the infrared imaging system, and
the composite image presenting unit is configured to present the composite image of the infrared moving image and the visible moving image.

5. The laser treatment apparatus of claim 4, wherein
the infrared imaging system is configured to capture the infrared moving image in parallel with capturing of the visible moving image by the imaging device, and
the composite image presenting unit is further configured to present the composite image in parallel with capturing of the infrared moving image and the visible moving image.

6. The laser treatment apparatus of claim 4, further comprising:
a movement mechanism configured to move the irradiation system, the illumination system, and the imaging system; and
a movement controller configured to control the movement mechanism based on the infrared moving image captured by the infrared imaging system to cause the irradiation system, the illumination system, and the imaging system to follow movement of the patient's eye.

7. The laser treatment apparatus of claim 1, wherein the composite image presenting unit is configured to present information indicating a site of interest of the patient's eye together with the composite image.

8. The laser treatment apparatus of claim 7, wherein
the composite image presenting unit includes a region of interest specifying unit configured to analyze the first image to specify a region of interest corresponding to the site of interest, and
the composite image presenting unit is further configured to present information indicating the region of interest together with the composite image.

9. The laser treatment apparatus of claim 8, wherein
the first image is a fluorescent contrast image, and
the region of interest specifying unit is configured to analyze the fluorescent contrast image to specify the region of interest corresponding to a lesion.

10. The laser treatment apparatus of claim 1, further comprising an observation system configured to guide the slit light returning from the patient's eye E to an ocular lens,
wherein the composite image presenting unit is further configured to guide the first image or the composite image to the ocular lens through an optical path of the observation system.

11. The laser treatment apparatus of claim 10, further comprising an operation unit,
wherein the composite image presenting unit is further configured to, based on output of the operation unit, switch between a state where the first image or the composite image is guided to the ocular lens and a state where the first image or the composite image is not guided to the ocular lens.

12. The laser treatment apparatus of claim 10, wherein
the observation system is a binocular observation system including a pair of ocular lenses, and
the composite image presenting unit is further configured to guide the first image or the composite image to the ocular lenses to be stereoscopically viewable through a pair of optical paths of the binocular observation system.

13. The laser treatment apparatus of claim 1, wherein the composite image presenting unit is further configured to display the composite image to be stereoscopically viewable on a display.
